(19) United States
(12) Patent Application Publication
(10) Pub. No.: US 2020/0038043 A1
(43) Pub. Date: Feb. 6, 2020

(54) MEDICAL SYSTEM

(71) Applicant: OLYMPUS CORPORATION, Tokyo (JP)

(72) Inventor: Kosuke MOTAI, Hidaka-shi (JP)

(73) Assignee: OLYMPUS CORPORATION, Tokyo (JP)

(21) Appl. No.: 16/594,395

(22) Filed: Oct. 7, 2019

Related U.S. Application Data
(63) Continuation of application No. PCT/JP2017/014634, filed on Apr. 10, 2017.

(51) Int. Cl. A61B 17/221; A61M 1/00; A61B 1/005; A61B 1/00

(52) U.S. Cl. CPC A61B 17/221; A61M 1/0023; A61B 2017/00358; A61B 1/00137; A61B 2017/2215; A61B 1/005

(57) ABSTRACT

A medical system including a grasp device having a longitudinal portion and an end effector attached to the longitudinal portion; an overtube having a collection lumen and a water supply lumen through which the longitudinal portion is inserted and having a distal end opening positioned inside the collection lumen; and an aspirator connected with the collection lumen, wherein the end effector further has a first member; and a second member mounted so as to open and close the end effector by moving relatively to the first member, wherein the second member forms an interior space between the first member and the second member for accommodating the tissues and has an opening communicating with the interior space when the end effector is closed, and the proximal end portion of the second member is enterable into the collection lumen.

### BACKGROUND

### Technical Field

The present invention relates to a medical system introduced to the inside of the body cavity for resecting and collecting the tissues.

This application is a continuation application based on a PCT International Application No. PCT/JP2017/014634, filed on Apr. 10, 2017. The content of the PCT International Application is incorporated herein by reference.

### Description of Related Art

The procedure of debriding necrotic tissue (Necrosectomy) in the pancreas is known as an endoscopic procedure.

In a case of performing the Necrosectomy by accessing to the pancreas from the stomach, an endoscope is introduced into an inner cavity such as a cyst generated in the pancreas by protruding the endoscope through a hole formed on the gastric parietal. Subsequently, various treatment devices such as grasping forceps, snares, and nets protruded from treatment tool channel of the endoscope are used to grasp the necrotic tissues. The grasped tissues are carried into the stomach and dropped in the stomach such that the grasped tissues are exhausted outside the body through the gastrointestinal tract.

Since a distal end portion of the endoscope has to be moved inside the stomach so as to drop the grasped tissues in the stomach, it is necessary for the endoscope to be moved between the pancreas and the stomach for several times to remove all of the necrotic tissues. Furthermore, the treatment tool has a size insertable through the treatment tool channel such that an amount of the necrotic tissues grasped during a cycle of the movement of the treatment tool is small. Thus, in many cases, it is necessary to perform a large cycles of reciprocal movements between the pancreas and the stomach until the procedures are finished.

The endoscopic Necrosectomy has an advantage of low invasiveness with respect to the Necrosectomy by the laparotomy procedures, however, the procedures of the endoscopic Necrosectomy become complicated such that extra one to two hours are necessary for removing all of the necrotic tissues due to the reasons shown above.

In PCT International Publication No. WO 2006-030596, a biopsy instrument is disclosed to include a hollow conduct which is configured to have a distal end portion having a collection unit configured to collect the biological tissues and a proximal end portion having an aspirator configured to remove the tissues collected by the collection unit. The tissues collected by the collection unit is recovered through the duct such that in the case when the Necrosectomy is performed by using the biopsy instrument disclosed in PCT International Publication No. WO 2006-030596 together with an endoscope, it is possible to continue the removal procedure of the necrotic tissues while avoiding the reciprocal movements of the endoscope between the stomach and the pancreas.

### SUMMARY

According to a first aspect of the present invention, a medical system is configured to have a grasp device, an overtube, and an aspirator, wherein the grasp device has an end effector configured to capture tissues and a longitudinal portion, the end effector being attached to a distal end of the longitudinal portion, the overtube has a collection lumen and a water supply lumen through which the longitudinal portion is inserted, the water supply lumen having a distal end opening positioned inside the collection lumen, and the aspirator is connected with the collection lumen. The end effector has a first member and a second member mounted so as to open and close the end effector by moving relatively to the first member, wherein the second member is configured to form an interior space between the first member for accommodating the tissues and the second member has an opening communicating with the interior space in a proximal end portion when the end effector is closed, and the proximal end portion of the second member is configured to be enterable into the collection lumen.

According to a second aspect of the present invention, in the medical system according to the first aspect, the first member and the second member may be rotatably supported by a rotary axial member, the opening communicating with the interior space when the end effector is closed may be formed in the proximal end portion of the first member, and the proximal end portion of the first member may be enterable into the collection lumen.

According to a third aspect of the present invention, in the medical system according to the first aspect, the grasp device may further have a sealing member attached in the vicinity of the end effector, the sealing member being configured to seal a gap between the end effector and an inner wall of the collection lumen when the proximal end portion of the first member enters in the collection lumen.

### BRIEF DESCRIPTION OF DRAWINGS

FIG. 1 is a view showing an overall configuration of a medical system according to a first embodiment of the present invention.

FIG. 2 is a front view showing a main body portion of a medical overtube.

FIG. 3 is a sectional view along I-I line in FIG. 2.

FIG. 4 is an enlarged sectional view showing a distal end portion of a grasp device.

FIG. 5 is a sectional view along II-II line in FIG. 4.

FIG. 6 is a view showing the grasp device passing through the medical overtube.

FIG. 7 is a view showing a process of an operation when using the medical system.

FIG. 8 is a view showing a process of an operation when using the medical system.

FIG. 9 is a view showing a process of an operation when using the medical system.

FIG. 10 is a view showing a process of an operation when using the medical system.

FIG. 11 is an enlarged view showing a distal end portion of a grasp device in a medical system according to a second embodiment of the present invention.

FIG. 12 is a sectional view showing the distal end portion.

FIG. 13 is a view showing a process of an operation when using the medical system.

FIG. 14 is a view showing a modification example of the grasp device.

FIG. 15 is a view showing a modification example of the grasp device.

FIG. 16 is a view showing modification examples of the grasp device and the overtube.

### DETAILED DESCRIPTION OF THE EMBODIMENTS

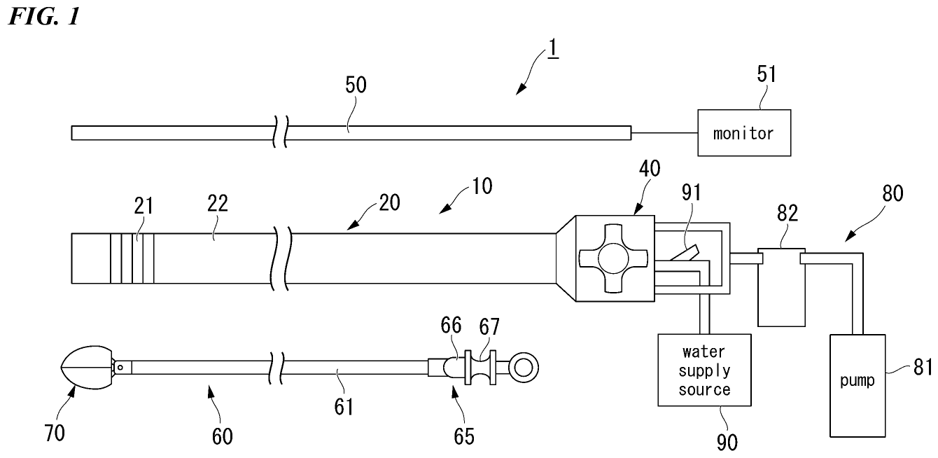

A first embodiment of the present invention will be described by referring to FIG. 1 to FIG. 10.

FIG. 1 is a view showing an example of a medical system 1 according to the present embodiment. The medical system. 1 has an overtube 10, an endoscope 50 and a grasp device 60 inserted through the overtube 10, and a suction mechanism (an aspirator) 80 and a water supply source 90 connected to the overtube 10.

The endoscope 50 is a so-called flexible endoscope having a flexible insertion portion. An image captured in a visual field of the endoscope 50 is shown on a monitor 51.

The endoscope 50 can be suitably selected from the conventional endoscopes in consideration of an outer diameter thereof.

The overtube 10 has a tubular flexible main body portion 20 and an operation portion 40 attached to the main body portion 20.

The main body portion 20 has an outer diameter capable of being inserted in to the body from the esophagus. For example, the outer diameter of the main body portion 20 is about 15 millimeters to 20 millimeters.

The main body portion 20 is formed from a flexible material such as the resin and the like, and the main body portion 20 has an active bending portion 21 disposed at a distal end side and a flexible tube portion 22 disposed at a proximal end side.

The active bending portion 21 has a conventional configuration including a plurality of nodal rings and the like, and the active bending portion 21 is connected to the operation portion 40 via a transmission member (not shown) such as a wire or the like. By operating the operation portion 40 to advance and retreat the transmission member in the longitudinal direction, the active bending portion 21 can be bent to a desired direction. With respect to the active bending portion 21, bendable directions (one direction, two directions, and four directions) can be suitably decided and a number of the transmission members and the specific configuration of the operation member 40 can be suitably selected from the conventional configurations in accordance with the bendable directions thereof.

The flexible tube portion 22 has a flexibility so as to be able to follow the travelling and the shape changes of the luminal organs.

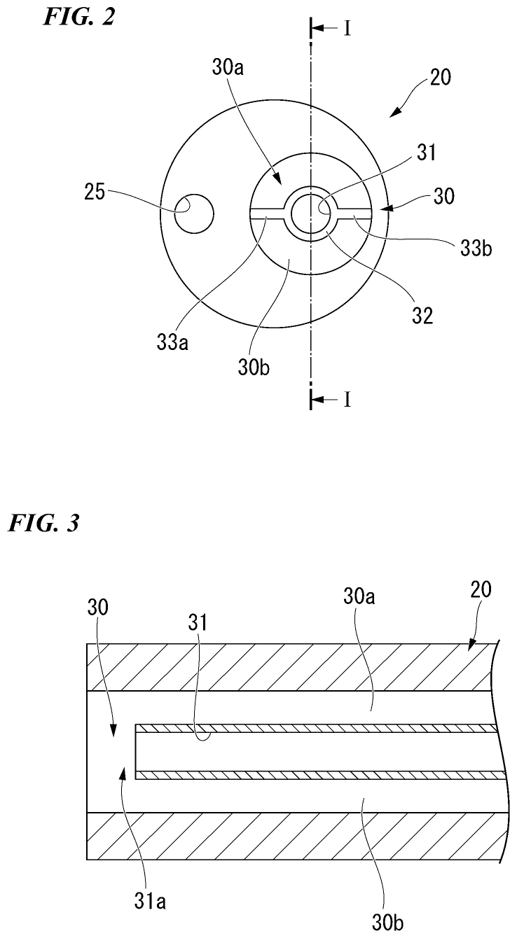

FIG. 2 is a front view of the main body portion 20, and FIG. 3 is a sectional view along I-I line in FIG. 2. As shown in FIGS. 2-3, a first lumen 25 through which the endoscope 50 is inserted and a second lumen (collection lumen) 30 for collecting the removed tissues are provided along the whole length of the main body portion 20.

A cross-sectional shape of the first lumen 25 is a circular shape, and an inner diameter of the first lumen 25 is a constant value along the whole length thereof. The value of the inner diameter of the first lumen 25 is decided such that the endoscope 50 can be inserted through the first lumen 25 with a predetermined clearance.

As shown in FIGS. 2-3, a water supply lumen 31 through which the grasp device 60 is inserted is disposed inside the second lumen 30. A distal end opening 31a of the water supply lumen 31 is disposed at a position spaced from the distal end of the main body portion 20 by several centimeters in the second lumen 30, for example. Partitions 33a, 33b are disposed at two positions in a circumferential direction of an outer circumference of a tube 32 configuring the water supply lumen 31, wherein the partitions 33a, 33b protrude outwardly in a radial direction and extend along the whole length of the tube 32.

The second lumen 30 is divided into a first conduit 30a and a second conduit 30b due to the tube 32 having the partitions 33a, 33b. The first conduit 30a and the second conduit 30b extend from the proximal end of the main body portion 20 in a non-communication state and the first conduit 30a and the second conduit 30b communicate with each other in front of the water supply lumen 31.

The grasp device 60 has a tubular flexible insertion portion 61, a treatment portion (an end effector) 70 provided at the distal end portion of the insertion portion 61, and an operation portion 65 provided at the proximal end portion of the insertion portion 61.

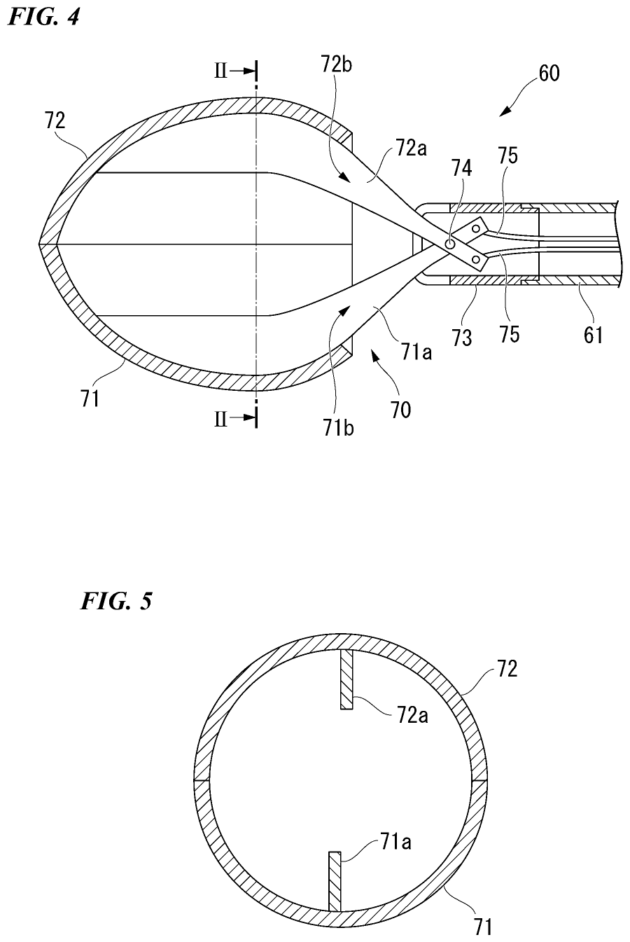

FIG. 4 is an enlarged sectional view showing the distal end portion of the grasp device 60. The treatment portion 70 has a pair of jaws as a first jaw (first member) 71 and a second jaw (second member) 72, and a support member 73 configured to support the pair of jaws 71, 72 such that the pair of jaws 71, 72 are rotatable. The support member 73 is fixed to the distal end portion of the insertion portion 61. The first jaw 71 and the second jaw 72 have links 71a, 72a respectively, and a rotary axial member 74 penetrating the links 71a, 72a is supported by the support member 73. Operation wires 75 are connected to the proximal end portions of the links 71a, 72a, respectively. Each operation wire 75 passes through the inside of the insertion portion 61 and extends until the operation portion 65.

The operation portion 65 has an operation portion main body 66 connected to the insertion portion 61 and a slider 67 attached to the operation portion main body 66 so as to be slidable. Each operation wire 76 extended to the operation portion 65 is connected to the slider 67. Accordingly, when the slider 67 is slided with respect to the operation portion main body 66, the operation wires 75 can be advanced or retracted inside the insertion portion 61. When the operation wires 75 are advanced or retracted, the pair of jaws 71, 72 rotate about the rotary axial member 74 as the center such that the treatment portion 70 opens and closes due to the relative movement of the pair of jaws 71, 72.

Either of the combination of the operation portion main body 66 and the insertion portion 61 and the combination of the slider 67 and the operation wires 75 is detachable. In other words, the grasp device 60 is configured to have the operation portion 65 to be attachable and detachable.

As shown in FIG. 4, the pair of jaws 71, 72 are configured to have openings 71b, 72b formed at the proximal end side thereof when the pair of jaws 71, 72 are closed at the distal end side. The pair of jaws 71, 72 have considerably larger dimensions than that of the jaws of the general grasp forceps, for example, the maximum dimension of the pair of jaws 71, 72 in the width direction (hereinafter described as W1) is about in a range from 15 to 20 millimeters.

FIG. 5 is a cross-sectional view along II-II line in FIG. 4. In the state in which the pair of jaws 71, 72 are closed, only the links 71a, 72a exist inside such that a wide interior space is generated therein. Accordingly, when compared with the general grasp forceps, far more amounts of tissues can be accommodated inside the pair of jaws 71, 72. Furthermore, in the state in which the pair of jaws 71, 72 are closed, the accommodated tissues and the like can be moved to the outside of the treatment portion 70 through the openings 71b, 72b communicating with the interior space.

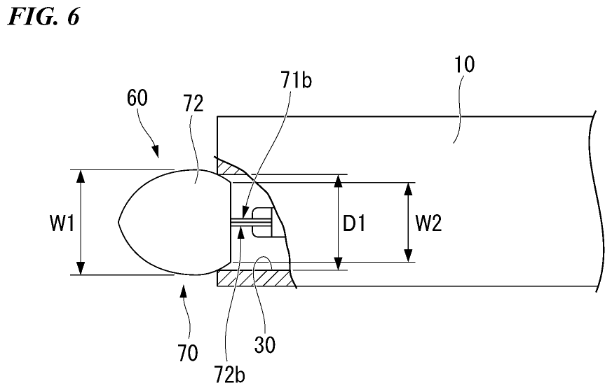

FIG. 6 is a view showing the grasp device 60 passing through the overtube 10. Since the maximum width dimension W1 of the pair of jaws 71, 72 in the width direction is larger than the inner diameter D1 of the second lumen 30, the whole of the treatment portion 70 cannot enter the second lumen 30. However, a width dimension W2 of the proximal end portion of the pair of jaws at which the openings 71b, 72b are formed is smaller than the inner diameter D1 such that the proximal end portion of the pair of jaws 71, 72 can enter the second lumen 30.

It is preferable to determine the above-described maximum width dimension W1 in the width direction such that the pair of jaws 71, 72 do not protrude outwardly from the overtube 10 in the radial direction in the state in which the proximal end portions of the pair of jaws 71, 72 are accommodated in the second lumen 30 as shown in FIG. 6.

The suction mechanism 80 is watertightly connected with each of the proximal openings of the first conduit 30a and the second conduit 30b. The suction mechanism 80 has a pump 81 configured to be able to perform the suction and supply air and a bottle 82 configured to store the solid and liquid collected due to the suction.

The water supply source 90 is connected to the proximal opening of the water supply lumen 31. A port 91, which is provided for the grasp device 60 to pass through, is disposed in a conduit between the water supply source 90 and the water supply lumen 31.

The operation when the medical system 1 having the above-described configuration according to the present embodiment is used, will be described using the example of the Necrosectomy performed in the pancreas.

Firstly, the surgeon confirms the position of the treatment target in the pancreas by an endoscope capable of performing both of the optical observation and the ultrasonic observation and establish an access path toward the treatment target by dissecting the gastric parietal and the pancreas. After the access path is established, the endoscope is pulled out and removed.

The surgeon inserts the endoscope 50 from the proximal opening of the first lumen 25 of the overtube 10. Further, the surgeon detaches the operation portion 65 from the grasp device 60, and the surgeon inserts the grasp device 60 into the second lumen 30 through the distal end opening thereof from the distal end side of the insertion portion 61. Subsequently, the surgeon attaches the operation portion 65 to the proximal end portion of the insertion portion 61, wherein the proximal portion of the insertion portion 61 protrudes from the port 91, so as to put the treatment portion 70 into the state in which the treatment portion 70 can be operated to open and close. Therefore, the preparation for initiating the medical system 1 is finished.

Next, the surgeon introduces the overtube 10, through which the endoscope 50 and the grasp device 60 are inserted, into the body of the patient. The surgeon observes the inside of the body using the endoscope 50 while operating the operation portion 40, if necessary, so as to move the overtube 10 to pass through the access path. As shown in FIG. 7, the surgeon finally introduces the distal end of the overtube 10 to the vicinity of the treatment target. In this example, the treatment target is in the pancreas Pc, and the necrotic tissues Nt which should be removed exist therein.

During the introduction procedures, air supply and water supply may be suitably performed using the second lumen 30 and the water supply lumen 31 respectively.

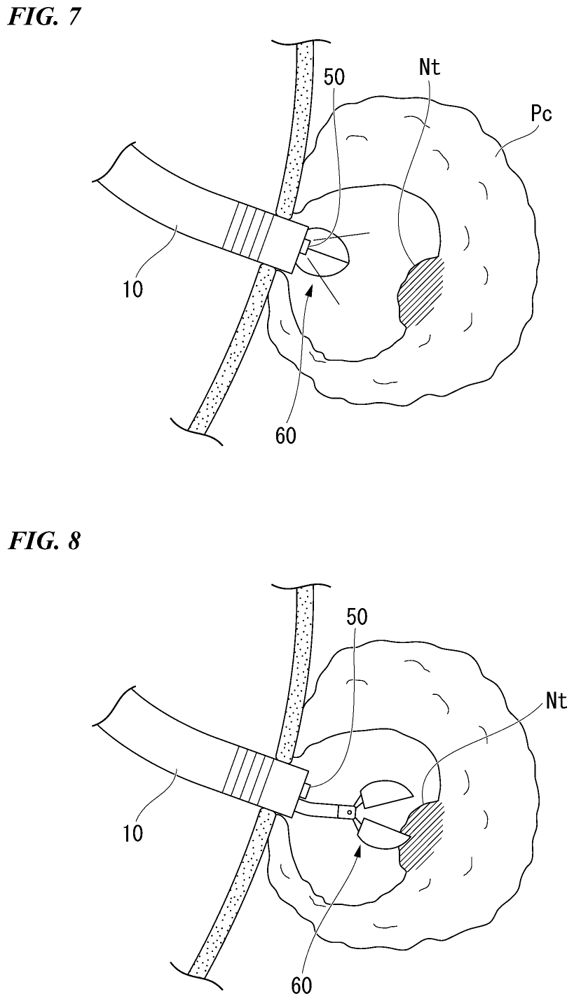

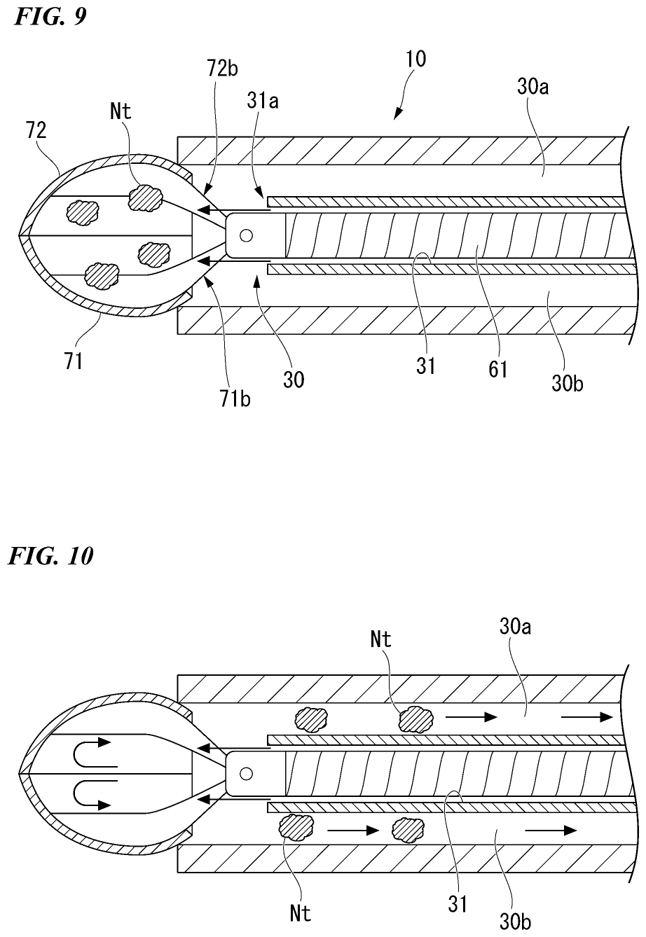

As shown in FIG. 8, the surgeon observes the treatment by the endoscope 50 while operating the grasp device 60 to grasp and remove the necrotic tissues Nt. The surgeon retracts the grasp device 60 grasping the necrotic tissues Nt, and as shown in FIG. 9, the surgeon moves the proximal end portion of the pair of jaws 71, 72 into the second lumen 30.

In the state shown in FIG. 9, a closed space in the medical system 1 is formed by the second lumen 30 and the interior space of the pair of jaws 71, 72.

When the surgeon supplies water in this state, the water passes through the interior of the water supply lumen 31 around the insertion portion 61, and as indicated by the arrows shown in FIG. 9, the water is ejected from the distal end opening 31a of the water supply lumen 31. The ejected water enters the interior space of the pair of jaws 71, 72 through the openings 71b, 72b so as to force the accommodated necrotic tissues Nt to be washed away. Further, once the surgeon operates the suction mechanism 80, a negative pressure is applied inside the first conduit 30a and the second conduit 30b such that the water and the necrotic tissues Nt flow into the first conduit 30a and the second conduit 30b. In other words, due to the water supply from the water supply lumen 31 and the suction through the first conduit 30a and the second conduit 30b, a circulating path for collecting the necrotic tissues Nt and the like which are grasped by the treatment portion 70 is established inside the second lumen 30.

The water and the necrotic tissues Nt which flow through the first conduit 30a and the second conduit 30b drop into the bottle 82 to be collected.

Thereafter, the surgeon advances the grasp devices 60 again to grasp the necrotic tissues Nt. Then, until all of the necrotic tissues Nt are removed, the same procedures are repeated. In the procedures shown above, the water supply and the suction may be performed simultaneously or alternatively.

According to the medical system 1 according to the present embodiment, the necrotic tissues Nt grasped by the grasp device 60 are collected by being moved into the second lumen 30 and sucked. Accordingly, the procedures of removing the necrotic tissues Nt can be continued while the overtube 10 and the endoscope 50 are maintained to be fixed. In other words, there is no necessity for the surgeon to move the overtube and the endoscope from the pancreas to the stomach every time when the surgeon grasps the necrotic tissues. As a result, the operation efficiency of the Necrosectomy procedures and the like can be remarkably improved.

Since the maximum width dimension W1 of the pair jaws 71, 72 of the grasp device 60 is larger than the inner diameter of the second lumen 30, compared with the general grasp forceps, it is far suitable to collect the tissues during every single operation. On the other hand, since the grasp device 60 has the opening 71b, 72b at the proximal end side and the width dimension W2 of the proximal end portion is smaller than the inner diameter of the second lumen 30, the grasped necrotic tissues and the like can be efficiently collected through the second lumen 30 only by moving the proximal end portion of the pair of jaws 71, 72 into the second lumen 30.

Furthermore, since the water supply lumen 31 is provided inside the second lumen 30, even in the case in which the viscosity of the necrotic tissues is high, the necrotic tissues can be washed away by the supplied water. Accordingly, it is possible to suitably avoid the situation in which the necrotic tissues cannot be collected since the necrotic tissues stick to the inner wall of the first conduit 30a and the second conduit 30b.

Additionally, since the insertion portion 61 of the grasp device 60 is inserted through the water supply lumen 31, the necrotic tissues and the like do not enter the water supply lumen 31. As a result, there is no adversely affect for the advance and retract operation of the grasp device 60 since the necrotic tissues and the like enter the water supply lumen 31.

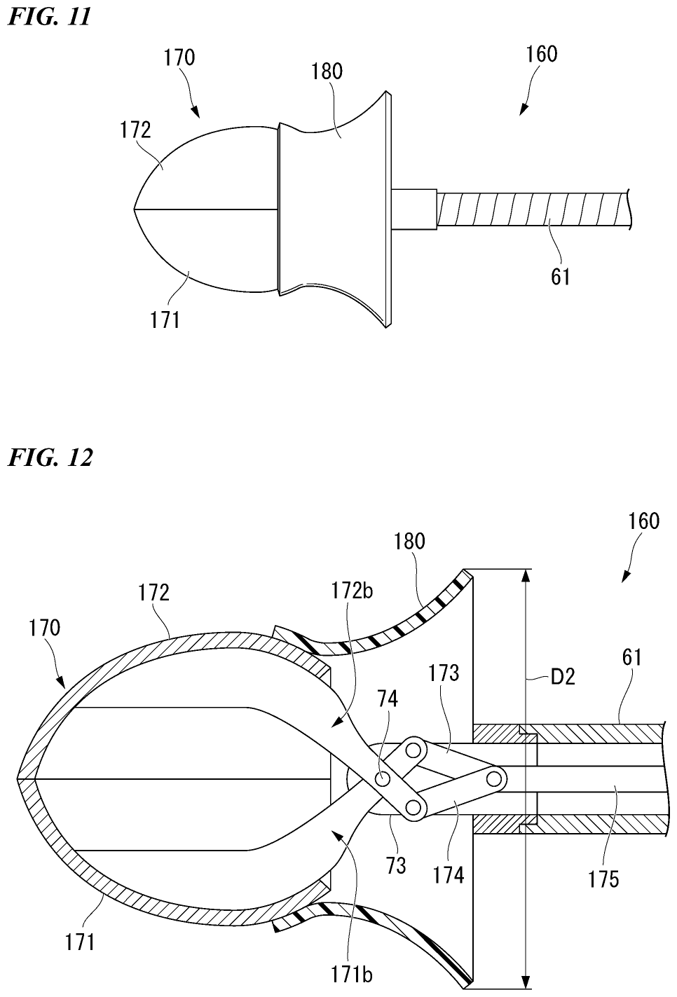

A second embodiment of the present invention will be described by referring to FIGS. 11-13. According to the present second embodiment, the structure of the grasp device is different from that of the first embodiment. In the following description, the configurations in common which have been described will be affixed with same numeral symbols and the duplicate description will be omitted.

FIG. 11 is an enlarged view showing a distal end portion of the grasp device 160 of the medical system according to the present embodiment. In the grasp device 160, a sealing member 180 having a skirt shape is attached to the proximal end side of the treatment portion 170.

FIG. 12 is a cross-sectional view of the distal end portion of the grasp device 160. The treatment portion 170 has the pair of jaws 171, 172 with the same shapes of those according to the first embodiment. The dimensions of the pair of jaws 171, 172 are smaller than those of the pair of jaws 71, 72 according to the first embodiment, and the treatment portion 170 is configured such that entire of the treatment tool 170 can enter the second lumen 30.

The proximal end portion of the pair of jaws 171, 172 are connected with links 173, 174 respectively, and the links 173, 174 are connected with each other by an operation wire 175. The second embodiment is similar with the first embodiment according to the configuration that opening 171b, 172b are provided at the proximal end portions of the pair of jaws 171, 172 respectively, and the configuration that the pair of jaws 171, 172 open and close when the operation wire 175 is advanced and retracted in the insertion portion 61 respectively.

The sealing member 180 is formed by elastic deformable material such as the rubber, the elastomer and the like, and the sealing member 180 is formed to have a tubular shape such that a diameter of the sealing member gradually increases toward the proximal end side of the sealing member 180. As described below, the sealing member 180 is preferably configured to be watertight so as to contribute to the formation of the closed space when the grasp device 160 is retracted. An outer dimension D2 of the proximal end portion of the sealing member 180 is larger than the inner diameter of the second lumen 30.

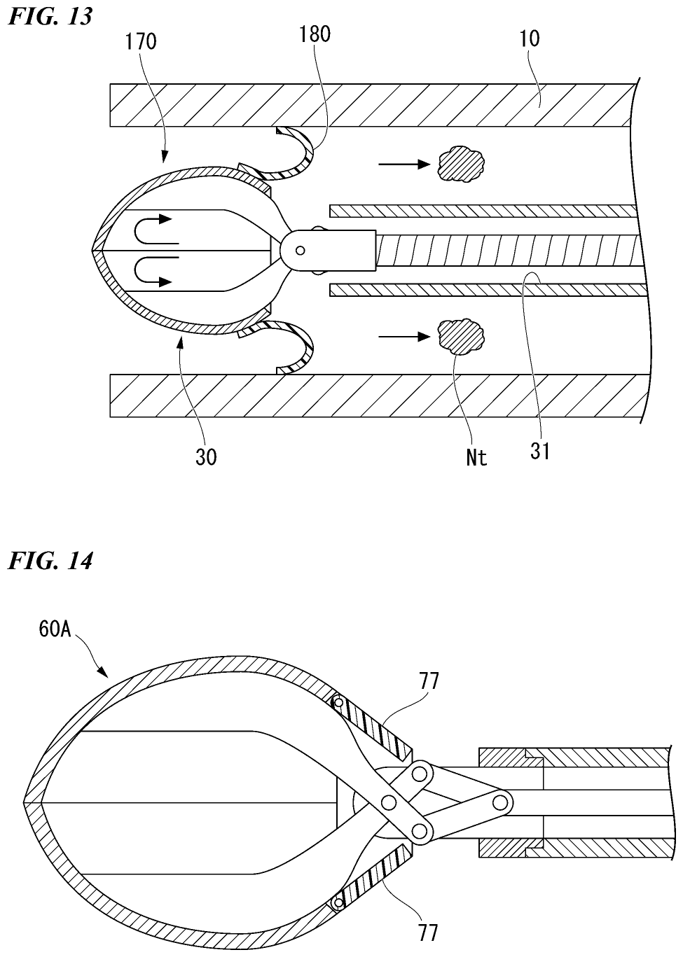

In the medical system according to the present embodiment, when the grasp device 160 is retracted after the necrotic tissues are grasped using the grasp device 160, the proximal end portion of the sealing member 180 abuts the distal end surface of the overtube 10 such that the proximal end portion of the sealing member 180 is turned over. When the grasp device 160 is further retracted, as shown in FIG. 13, the entire of the treatment portion 170 moves to the inside of the second lumen 30. A gap is generated between the treatment portion 170 and the inner wall of the second lumen 30, however, the gap is sealed over the whole circumference by the deformed sealing member 180. Accordingly, both of the formation of the closed space and the establishment of the circulating path for collecting the necrotic tissues are achieved.

Other configurations of the second embodiment are substantially the same with those of the first embodiment.

According to the medical system having the grasp device 160 according to the present embodiment, as same as the first embodiment, the operation efficiency of the Necrosectomy procedures and the like can be remarkably improved.

The above-described sealing member 180 may be attached to the grasp device 60 according to the first embodiment. Once the sealing member 180 is provided, it is possible to form the closed space without pushing the pair of jaws 71, 72 strongly against the overtube 10 so as to make the operation easy.

The embodiments of the invention have been described above with reference to the drawings, but specific structures of the invention are not limited to the embodiments and may include various modifications without departing from the scope of the invention. The invention is not limited to the above-mentioned embodiments and is limited only by the accompanying claims.

For example, as similar as the grasp device 60A shown in the modification example in FIG. 14, covers 77 may be provided on the openings at the proximal end side of the pair of jaws. According to such a configuration, leakage of the necrotic tissues and the like which are grasped and accommodated in the treatment portion can be prevented before the collection. The operation procedures of opening the cover 77 can be suitably determined by providing an open/close wire connected to the cover and extended to the operation portion through the inside of the insertion portion 61, or generating the negative pressure in the closed space.

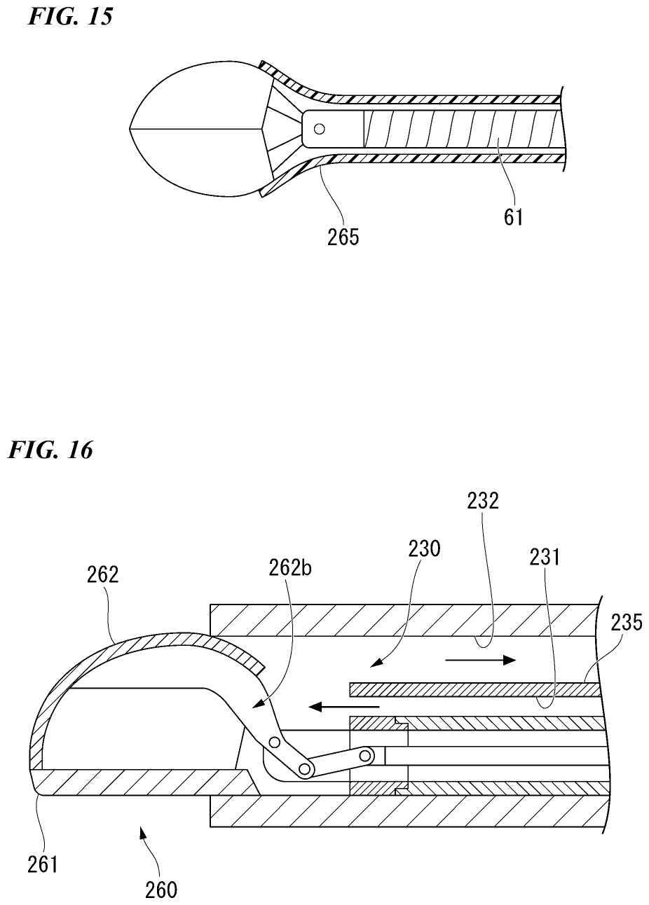

As shown in FIG. 15, instead of the cover, the grasp device may be configured to have an outer sheath 265 in the insertion portion 61 such that the openings at the proximal end side of the pair of jaws can be covered when the outer sheath 265 is advanced relatively to the insertion portion 61. Additionally, in order to make FIG. 15 easy to view, only the outer sheath 265 is shown in a cross-sectional view.

The grasp device 60 is not limited to the configuration in which both of the pair of jaws are rotated to open and close the grasp device 60. For example, as the grasp device 260 shown in FIG. 16, the grasp device may be configured to have a single opening configuration in which only the jaw 262 between the jaw (first member) 261 and the jaw (second member) 262 at the distal end portion thereof has the opening 262b and is moveable. In this case, as shown in FIG. 16, the configuration in which the interior of the second lumen 230 is divided into the water supply lumen 231 and the collection conduit 232 by a single partition 235 may be selected.

Additionally, in the medical system according to the present invention, it is not necessary for the overtube to have flexibility. For example, in the case of inserting the overtube into the body cavity such as the abdominal cavity and the thorax via a trocar and the like, the overtube may be configured as a rigid overtube such that the main body portion is formed by the rigid material and only the active bending portion is bendable.

An example of the surgical procedures performed inside the body cavity can be given as the collection and removal of tissue fragments separated in the abdominal cavity after using the morcellator during the laparoscopic myomectomy.

What is claimed is:

1. A medical system, comprising:
a grasp device having an end effector configured to capture tissues and a longitudinal portion, the end effector being attached to a distal end of the longitudinal portion;
an overtube having a collection lumen and a water supply lumen through which the longitudinal portion is inserted, the water supply lumen having a distal end opening positioned inside the collection lumen; and
an aspirator connected with the collection lumen,
wherein the end effector further comprises:
a first member; and
a second member mounted so as to open and close the end effector by moving relatively to the first member,
wherein the second member is configured to form an interior space between the first member and the second member for accommodating the tissues, and the second member has an opening communicating with the interior space in a proximal end portion when the end effector is closed, and
wherein the proximal end portion of the second member is configured to be enterable into the collection lumen.

2. The medical system according to claim 1,
wherein the first member and the second member are rotatably supported by a rotary axial member,
wherein the opening communicating with the interior space when the end effector is closed is formed in the proximal end portion of the first member, and
wherein the proximal end portion of the first member is enterable into the collection lumen.

3. The medical system according to claim 1,
wherein the grasp device further has a sealing member attached in the vicinity of the end effector, the sealing member being configured to seal a gap between the end effector and an inner wall of the collection lumen when the proximal end portion of the first member enters in the collection lumen.
